(19) United States
(12) Patent Application Publication
(10) Pub. No.: US 2025/0327131 A1
(43) Pub. Date: Oct. 23, 2025

(54) METHODS FOR IDENTIFYING CANCER IN A SUBJECT

(71) Applicant: The Johns Hopkins University, Baltimore, MD (US)

(72) Inventors: Christopher Douville, Baltimore, MD (US); Bert Vogelstein, Baltimore, MD (US); Kenneth W. Kinzler, Frankford, DE (US); Nickolas Papadopoulos, Towson, MD (US); Samuel Curtis, Baltimore, MD (US)

(21) Appl. No.: 18/865,468

(22) PCT Filed: May 12, 2023
(86) PCT No.: PCT/US2023/022077

§ 371 Date: Nov. 13, 2024

Related U.S. Application Data
(60) Provisional application No. 63/341,846, filed on May 13, 2022.

(51) Int. Cl. C12Q 1/6886; C12Q 1/6874

(52) U.S. Cl. CPC C12Q 1/6886; C12Q 1/6874

(57) ABSTRACT

Provided herein are methods of identifying a subject as having a disease, the method comprising: (a) obtaining a biological sample from the subject, wherein the biological sample comprises cell-free DNA (cfDNA), wherein the cfDNA comprises a plurality of cfDNA fragments; (b) determining an end sequence of a cfDNA fragment of the plurality of cfDNA fragments; (c) determining a level of GC content of the cfDNA fragment; and (d) analyzing the determined end sequence and the level of GC content of the cfDNA fragment, thereby identifying the subject as having the disease by determining a relationship between the determined end sequence and the level of GC content of the cfDNA fragment.

### CROSS-REFERENCE TO RELATED APPLICATIONS

This application claims priority to U.S. Provisional Patent Application No. 63/341,846, filed on May 13, 2022, which is incorporated herein by reference in its entirety.

### FEDERALLY SPONSORED RESEARCH OR DEVELOPMENT

This invention was made with government support under grant CA062924, GM136577, GM135083 and CA006973 awarded by the National Institutes of Health. The government has certain rights in the invention.

### TECHNICAL FIELD

The present disclosure relates to the area of nucleic acid analysis. In particular, it relates to nucleic acid sequence analysis which can determine an end sequence of cell-free DNA (cfDNA) from a subject and identify the subject as having cancer.

### BACKGROUND

The earlier detection of cancer could lead to substantial reductions in morbidity and mortality because all known cancer treatments are more successful when there's a lower tumor burden in the patient. The evaluation of cell-free DNA (cfDNA) from plasma is one of the most promising approaches for such earlier detection. Numerous ways to use cfDNA have been described in the literature. Genetic alterations in cfDNA—such as mutations or copy number alterations—have been extensively used for this purpose. Epigenetic alterations, in particular changes in DNA methylation, have also been used to correctly classify patients with cancer. Other types of epigenetic changes, reflecting chromatin organization rather than covalent changes in DNA, have more recently gained attention. Because DNA is always wrapped in nucleosomes, whether in the cell or in the circulation, changes in chromatin structure result in changes of the fragments produced by nucleases in the cell of origin or in the circulation. This gives rise to different patterns of fragmentation with respect to gene regulatory elements such as nucleosome positioning in promoters and enhancers as well as differences in fragment sizes or the sequences at the ends of fragments. Because epigenetics, rather than genetics, is responsible for the differences in cell types, these patterns, as well as methylation patterns, can reveal the cell of origin of the fragments including the cancer cell of origin.

Though the results to date of these cfDNA-based technologies are promising, further research to increase sensitivity of detection of cancer patients while maintaining high specificity is a high research and clinical priority.

### SUMMARY

Provided herein are methods of identifying a subject as having a disease, the methods including: (a) obtaining a biological sample from the subject, wherein the biological sample comprises cell-free DNA (cfDNA), wherein the cfDNA comprises a plurality of cfDNA fragments; (b) determining an end sequence of a cfDNA fragment of the plurality of cfDNA fragments; (c) determining a level of GC content of the cfDNA fragment; and (d) analyzing the determined end sequence and the level of GC content of the cfDNA fragment, thereby identifying the subject as having the disease by determining a relationship between the determined end sequence and the level of GC content of the cfDNA fragment.

In some embodiments, the cell-free DNA comprises double stranded DNA.

In some embodiments, the determined end sequence is at the 5′-end of one or both strands of the cfDNA. In some embodiments, the determined end sequence is 2, 3, 4, 5, or 6 bases in length. In some embodiments, the determined end sequence is 3 bases in length. In some embodiments, the determined end sequences comprises TGT, GAG, GCG, or ATT.

In some embodiments, the relationship of the determined end sequence and the level of GC content of the cfDNA fragment from a subject having the disease is different as compared to the relationship of the determined end sequence and the level of GC content of the cfDNA fragment from a subject that does not have the disease.

In some embodiments, the biological sample is a plasma sample. In some embodiments, the biological sample is a cerebrospinal fluid (CSF) sample.

In some embodiments, the disease is a cancer. In some embodiments, the cancer is a cancer of the central nervous system. In some embodiments, the cancer is a metastatic lesion. In some embodiments, the cancer is selected from bladder cancer, breast cancer, cervical cancer, colorectal cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, the cancer is a pancreatic cancer, lung cancer, or colorectal cancer.

Also provided herein are methods of identifying a relationship between an end sequence and a disease state in a subject, the methods including: (a) obtaining a first cfDNA sample from a first subject having a disease and a second cfDNA sample from a second subject that does not have the disease, wherein the cfDNA samples comprise a plurality of cfDNA fragments; (b) determining an end sequence of a first cfDNA fragment of the plurality of first cfDNA fragments and determining a first level of GC content of the first cfDNA fragment; (c) determining the same end sequence of a second cfDNA fragment of the plurality of second cfDNA fragments and determining a second level of GC content of the second cfDNA fragment; (d) measuring a first frequency of the determined end sequence in the first cfDNA fragment and a second frequency of the same determined end sequence in the second cfDNA fragment; (e) identifying a first relationship between the first frequency of the determined end sequence and the determined first level of GC content from the first subject; (f) identifying a second relationship between the second frequency of the determined end sequence and the determined second level of GC content from the second subject; and (g) determining that the first relationship is indicative of the disease and that the second relationship is not indicative for the disease state.

In some embodiments, a cfDNA fragment comprises double stranded DNA.

In some embodiments, the determined end sequence is at the 5′-end of one or both strands of the cfDNA fragment. In some embodiments, the determined end sequence is 2, 3, 4, 5, or 6 bases in length. In some embodiments, the determined end sequence is 3 bases in length. In some embodiments, the determined end sequence comprises TGT, GAG, GCG, or ATT.

In some embodiments, the first frequency of the determined end sequence is indicative of the presence of a somatic mutation when the first relationship is indicative of the disease. In some embodiments, the first frequency of the determined end sequence is indicative of the presence of a copy number variation when the first relationship is indicative of the disease.

In some embodiments, the disease is a cancer. In some embodiments, the cancer is a cancer of the central nervous system. In some embodiments, the cancer is a metastatic lesion. In some embodiments, the cancer is selected from bladder cancer, breast cancer, cervical cancer, colorectal cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, the cancer is a pancreatic cancer, lung cancer, or colorectal cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1J show graphs of the frequencies of trimers at the 5′-ends of cfDNA fragments according to the GC content of the entire fragment. The trend of frequency vs. GC for each trimer can be roughly predicted using the GC content of the trimer itself. FIG. 1A shows data for the end sequence TGT from patients in Cohorts 1 and 2. FIG. 1B shows data for the end sequence TGT from patients in Cohort 3. FIG. 1C shows data for the end sequence GAG from patients in Cohorts 1 and 2. FIG. 1D shows data for the end sequence GAG from patients in Cohort 3. FIG. 1E shows data for the end sequence GCG from patients in Cohorts 1 and 2. FIG. 1F shows data for the end sequence GCG from patients in Cohort 3. FIG. 1G shows data for the end sequence ATT from patients in Cohorts 1 and 2. FIG. 1H shows data for the end sequence ATT from patients in Cohort 3. FIG. 1I shows data for the end sequence CGA from patients in Cohorts 1 and 2. FIG. 1J shows data for the end sequence CGA from patients in Cohort 3.

FIGS. 2A-2F are graphs showing the comparison of average z-score for trimers in GC bins of 1% between data generated during the study (Cohort 1 and 2; 106 samples). FIG. 2A shows data for the end sequence TGT. FIG. 2B shows data for the end sequence GAG. FIG. 2C shows data for the end sequence GCG. FIG. 2D shows data for the end sequence ATT. FIG. 2E shows data for the end sequence CGA from patients in Cohort 1 and 2. FIG. 2F shows data for the end sequence CGA from patients in Cohort 3.

FIGS. 3A-3F are graphs showing performance of model on training set measured using 5-fold cross-validation (CV). FIG. 3A shows data where 5-Fold CV was performed ten times and average sample score are displayed (Cohort 1, 22 cancer 37 healthy). FIG. 3B shows ROC curves that were calculated for each iteration of 5-Fold CV and the averages and standard deviation between iterations is displayed (Cohort 1, 22 cancer 37 healthy). FIG. 3C shows data where a model trained on Cohort 1 (22 cancer, 37 healthy) was tested on an unseen test set (Cohort 2, 21 cancers 27 healthy). FIG. 3D shows ROC curves that were calculated using scores from FIG. 3C (Cohort 2, 21 cancers 27 healthy). FIG. 3E shows data where 5-Fold CV was performed ten times and average sample score are displayed (Cohort 3, 144 cancer (including pancreatic, lung, and colorectal cancers) and 208 healthy). FIG. 3F shows ROC curves that were calculated for each iteration of 5-Fold CV and the averages and standard deviation between iterations is displayed (Cohort 3, 144 cancer (including pancreatic, lung, and colorectal cancers) and 208 healthy).

FIGS. 4A-4B are graphs showing comparison of Modified End-based sequencing (MendSeqS) and End-based sequencing (EndSeqS) at various levels of downsampling. Randomized downsampling was performed three times and the average and standard deviation for each depth are shown.

FIGS. 5A-5B show 1152 features chosen with Mann-Whitney U test (MWU) p-values>70^{th} percentile determined using Cohort 1. Y-axis illustrates how many features of a given GC bin were included within the feature set.

FIGS. 6A-6B show a workflow of the Reference Normalization method, wherein results show a decreased coefficient of variation (CoV), increased Z-Score, and more significant p-value in the majority of the features that were normalized with reference normalization.

FIGS. 7A-7B are graphs showing that MendSeqS features were more statistically significant compared to EndSeqS features when comparing patients with cancer to healthy individuals. Using a high signal-to-noise “Discovery Set”, it was illustrated that the fragment end-motifs are significantly correlated with the neoplastic content in the plasma.

FIGS. 8A-8B show a workflow of feature selection. FIG. 8A shows step one of removing noise using a panel of normals, and FIG. 8B shows step two of SaferSeqS-informed feature selection using the “Discovery Cohort.”

FIGS. 9A-9C show that an optimized MendSeqS model (3 features) effectively detects early and late-stage cancers. FIG. 9A shows results of the model effectively detecting early and late-stage cancers using only three features in cross-validation of the training set. FIG. 9B shows results of the model effectively detecting early and late-stage cancers using only three features in an independent validation set. FIG. 9C shows independent validation scores with specificity thresholds determined using the training set.

### DETAILED DESCRIPTION

Several characteristics of cell-free DNA (cfDNA) in the plasma have been shown to be associated with neoplasia and the evaluation of the cfDNA from plasma is one of the most promising approaches for earlier detection of such neoplasia.

Provided herein are methods of identifying a subject as having a disease, the method including: (a) obtaining a biological sample from the subject, wherein the biological sample comprises cell-free DNA (cfDNA), wherein the cfDNA comprises a plurality of cfDNA fragments; (b) determining an end sequence of a cfDNA fragment of the plurality of cfDNA fragments; (c) determining a level of GC content of the cfDNA fragment; and (d) analyzing the determined end sequence and the level of GC content of the cfDNA fragment, thereby identifying the subject as having the disease by determining a relationship between the determined end sequence and the level of GC content of the cfDNA fragment.

Also provided herein are methods of method of identifying a relationship between an end sequence and a disease state in a subject, the method including: (a) obtaining a first cfDNA sample from a first subject having a disease and a second cfDNA sample from a second subject that does not have the disease, wherein the cfDNA samples comprise a plurality of cfDNA fragments; (b) determining an end sequence of a first cfDNA fragment of the plurality of first cfDNA fragments and determining a first level of GC content of the first cfDNA fragment; (c) determining the same end sequence of a second cfDNA fragment of the plurality of second cfDNA fragments and determining a second level of GC content of the second cfDNA fragment; (d) measuring a first frequency of the determined end sequence in the first cfDNA fragment and a second frequency of the same determined end sequence in the second cfDNA fragment; (e) identifying a first relationship between the first frequency of the determined end sequence and the determined first level of GC content from the first subject; (f) identifying a second relationship between the second frequency of the determined end sequence and the determined second level of GC content from the second subject; and (g) determining that the first relationship is indicative of the disease and that the second relationship is not indicative for the disease state.

Various non-limiting aspects of these methods are described herein, and can be used in any combination without limitation. Additional aspects of various components of methods for identifying the presence or absence of a mutation and methylation are known in the art.

It must be noted that, as used in the specification and the appended claims, the singular forms “a,” “an” and “the” include plural referents unless the context clearly dictates otherwise.

As used herein, the terms “cancer”, “malignancy”, “neoplasm”, “tumor”, and “carcinoma”, refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

As used herein, “nucleic acid” is used to refer to any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)). A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

As used herein, the term “subject” is intended to refer to any mammal. In some embodiments, the subject is cat, a dog, a goat, a human, a non-human primate, a rodent (e.g., a mouse or a rat), a pig, or a sheep. In some embodiments, a subject is suffering from a relevant disease, disorder or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### Method of Identifying Cancer in a Subject

Provided herein are methods of identifying a subject as having a disease that include (a) obtaining a biological sample from the subject, wherein the biological sample comprises cell-free DNA (cfDNA), wherein the cfDNA comprises a plurality of cfDNA fragments; (b) determining an end sequence of a cfDNA fragment of the plurality of cfDNA fragments; (c) determining a level of GC content of the cfDNA fragment; and (d) analyzing the determined end sequence and the level of GC content of the cfDNA fragment, thereby identifying the subject as having the disease by determining a relationship between the determined end sequence and the level of GC content of the cfDNA fragment.

As used herein, a “cell-free DNA” can refer to non-encapsulated DNA that is released from cells into the circulatory system throughout the body. Cell-free DNA (cfDNA) includes nucleic acid fragments that enter the bloodstream during apoptosis or necrosis of cells. cfDNA can be found in plasma and other body fluids (e.g., cerebral spinal fluid (CSF), pleural fluid, urine, and saliva). Previous studies indicated that most of the plasma cfD.NA molecules originate from the hematopoietic system in healthy individuals. However, in certain physiological or pathological conditions, such as pregnancy, organ transplantation, and cancers, the related/affected tissues could release additional DNA into peripheral circulation. Therefore, detection of cfDNA in peripheral blood could identify abnormalities of individuals in a noninvasive manner.

In some embodiments, the cfDNA comprises double stranded DNA. In some embodiments, the cfDNA comprises one or more cfDNA fragments.

In some embodiments, cfDNA can be about 50 to about 450 (e.g., about 50 to about 400, about 50 to about 350, about 50 to about 300, about 50 to about 250, about 50 to about 200, about 50 to about 150, about 50 to about 100, about 100 to about 450, about 100 to about 400, about 100 to about 350, about 100 to about 300, about 100 to about 250, about 100 to about 200, about 100 to about 150, about 150 to about 450, about 150 to about 400, about 150 to about 350, about 150 to about 300, about 150 to about 250, about 150 to about 200, about 200 to about 450, about 200 to about 400, about 200 to about 350, about 200 to about 300, about 200 to about 250, about 250 to about 450, about 250 to about 400, about 250 to about 350, about 250 to about 300, about 300 to about 450, about 300 to about 400, about 300 to about 350, about 350 to about 450, about 350 to about 400, or about 400 to about 450) nucleotides in length.

In some embodiments, an end sequence comprises a sequence at an end of a cfDNA fragment. In some embodiments, an end sequence comprises a sequence at an end of a cfDNA fragment, wherein the end sequence is 2, 3, 4, 5, or 6 bases in length. In some embodiments, an end sequence can be at the 5′-end of a cfDNA fragment. In some embodiments, an end sequence can be at the 3′-end of a cfDNA fragment.

In some embodiments, an end sequence at an end of a cfDNA fragment can be determined. In some embodiments, an end sequence at an end of one or more cfDNA fragments can be determined. In some embodiments, the one or more determined end sequences are at the 5′-end of one or both strands of the cfDNA. In some embodiments, the one or more determined end sequences are at the 3′-end of one or both strands of the cfDNA. In some embodiments, the one or more determined end sequences are 2, 3, 4, 5, or 6 bases in length. In some embodiments, the one or more determined end sequences are 2 bases in length. In some embodiments, the one or more determined end sequences are 3 bases in length. In some embodiments, the one or more determined end sequences are 4 bases in length.

In some embodiments, a subject is identified as having a disease based on analysis of one or more end sequences. In some embodiments, a subject is identified as having a disease based on analysis of 2, 3, 4, 5, 6, 7, 8, 9, 10, or more end sequences. In some embodiments, a subject is identified as having a disease based on analysis of a single end sequence.

In some embodiments, at least one or more of the determined end sequences can comprise TTT, TTC, TTA, TTG, CTT, CTC, CTA, CTG, ATT, ATC, ATA, ATG, GTT, GTC, GTA, GTG, TCT, TCC, TCA, TCG, CCT, CCC, CCA, CCG, ACT, ACC, ACA, ACG, GCT, GCC, GCA, GCG, TAT, TAC, TAA, TAG, CAT, CAC, CAA, CAG, AAT, AAC, AAA, AAG, GAT, GAC, GAA, GAG, TGT, TGC, TGA, TGG, CGT, CGC, CGA, CGG, AGT, AGC, AGA, AGG, GGT, GGC, GGA, or GGG. In some embodiments, at least one or more of the determined end sequences comprises TGT, GAG, GCG, or ATT.

As used herein, a “level of GC content” or “GC content” can refer to the percentage of nitrogenous bases in a DNA or RNA molecule that are either guanine (G) or cytosine (C). The level of GC content indicates the proportion of G and C bases out of an implied four bases, also including adenine (A) and thymine (T) in DNA and adenine (A) and uracil (U) in RNA. In some embodiments, the level of GC content can be measured for a fragment of a DNA. In some embodiments, the level of GC content can be measured for an entire genome

In some embodiments, the relationship of the determined end sequence and the level of GC content of the cfDNA fragment from a subject having the disease is different as compared to the relationship of the determined end sequence and the level of GC content of the cfDNA fragment from a subject that does not have the disease.

In some embodiments, the cfDNA can be obtained from a biological sample. The biological sample may be obtained from a subject. In some embodiments, the subject is a mammal. Examples of mammals from which the cfDNA can be obtained and used in the methods described herein include, without limitation, humans, non-human primates (e.g., monkeys), dogs, cats, sheep, rabbits, mice, hamsters, and rats. In some embodiments, the subject is a human subject.

As used herein, biological samples can include but are not limited to plasma, serum, blood, tissue, tumor sample, stool, sputum, saliva, urine, sweat, tears, ascites, bronchoaveolar lavage, semen, archeologic specimens and forensic samples. In some embodiments, the biological sample is a solid biological sample (e.g., a tumor sample). In some embodiments, the biological sample is a liquid biological sample. Liquid biological samples can include, but are not limited to plasma, serum, blood, sputum, saliva, urine, sweat, tears, ascites, bronchoaveolar lavage, and semen. In some embodiments, the liquid biological sample is cell free or substantially cell free. In some embodiments, the biological sample is a plasma or serum sample. In some embodiments, the liquid biological sample is a whole blood sample. In some embodiments, the liquid biological sample comprises peripheral mononuclear blood cells. In some embodiments, the biological sample is a cerebrospinal fluid (CSF) sample.

In some embodiments, a nucleic acid sample (e.g., cfDNA) has been isolated and purified from the biological sample. Nucleic acid can be isolated and purified from the biological sample using any means known in the art. For example, a biological sample may be processed to separate nucleic acids from unwanted components of the biological sample (e.g., proteins, cell walls, other contaminants). For example, nucleic acid can be extracted from the biological sample using liquid extraction (e.g., Trizol, DNAzol) techniques. Nucleic acid can also be extracted using commercially available kits (e.g., Qiagen DNeasy kit, QIAamp kit, Qiagen Midi kit, QIAprep spin kit).

In some embodiments, the methods described herein can be used to identify a subject as having a disease. In some embodiments, the disease is a cancer. In some embodiments, the cancer is a cancer of the central nervous system. In some embodiments, the cancer is a metastatic lesion. In some embodiments, the cancer can be bladder cancer, breast cancer, cervical cancer, colorectal cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma or prostate cancer. In some embodiments, the cancer is a pancreatic cancer, lung cancer, or colorectal cancer.

### Method of Identifying a Correlation Between an End Sequence and GC Content in cfDNA

Provided herein are methods of method of identifying a relationship between an end sequence and a disease state in a subject that include (a) obtaining a first cfDNA sample from a first subject having a disease and a second cfDNA sample from a second subject that does not have the disease, wherein the cfDNA samples comprise a plurality of cfDNA fragments; (b) determining an end sequence of a first cfDNA fragment of the plurality of first cfDNA fragments and determining a first level of GC content of the first cfDNA fragment; (c) determining the same end sequence of a second cfDNA fragment of the plurality of second cfDNA fragments and determining a second level of GC content of the second cfDNA fragment; (d) measuring a first frequency of the determined end sequence in the first cfDNA fragment and a second frequency of the same determined end sequence in the second cfDNA fragment; (e) identifying a first relationship between the first frequency of the determined end sequence and the determined first level of GC content from the first subject; (f) identifying a second relationship between the second frequency of the determined end sequence and the determined second level of GC content from the second subject; and (g) determining that the first relationship is indicative of the disease and that the second relationship is not indicative for the disease state.

In some embodiments, the cfDNA can comprise double stranded DNA. In some embodiments, the cfDNA comprises one or more cfDNA fragments.

In some embodiments, cfDNA fragments in a cfDNA sample can be about 50 to about 450 (e.g., about 50 to about 400, about 50 to about 350, about 50 to about 300, about 50 to about 250, about 50 to about 200, about 50 to about 150, about 50 to about 100, about 100 to about 450, about 100 to about 400, about 100 to about 350, about 100 to about 300, about 100 to about 250, about 100 to about 200, about 100 to about 150, about 150 to about 450, about 150 to about 400, about 150 to about 350, about 150 to about 300, about 150 to about 250, about 150 to about 200, about 200 to about 450, about 200 to about 400, about 200 to about 350, about 200 to about 300, about 200 to about 250, about 250 to about 450, about 250 to about 400, about 250 to about 350, about 250 to about 300, about 300 to about 450, about 300 to about 400, about 300 to about 350, about 350 to about 450, about 350 to about 400, or about 400 to about 450) nucleotides in length.

In some embodiments, an end sequence comprises a sequence at an end of a cfDNA fragment. In some embodiments, an end sequence comprises a sequence at an end of a cfDNA fragment, wherein the end sequence is 2, 3, 4, 5, or 6 bases in length. In some embodiments, an end sequence can be at the 5′-end of a cfDNA fragment. In some embodiments, an end sequence can be at the 3′-end of a cfDNA fragment.

In some embodiments, an end sequence at an end of a cfDNA fragment can be determined. In some embodiments, an end sequence at an end of one or more cfDNA fragments can be determined. In some embodiments, the one or more determined end sequences are at the 5′-end of one or both strands of the cfDNA. In some embodiments, the one or more determined end sequences are at the 3′-end of one or both strands of the cfDNA. In some embodiments, the one or more determined end sequences are 2, 3, 4, 5, or 6 bases in length. In some embodiments, the one or more determined end sequences are 2 bases in length. In some embodiments, the one or more determined end sequences are 3 bases in length. In some embodiments, the one or more determined end sequences are 4 bases in length.

In some embodiments, at least one or more of the determined end sequences can comprise TTT, TTC, TTA, TTG, CTT, CTC, CTA, CTG, ATT, ATC, ATA, ATG, GTT, GTC, GTA, GTG, TCT, TCC, TCA, TCG, CCT, CCC, CCA, CCG, ACT, ACC, ACA, ACG, GCT, GCC, GCA, GCG, TAT, TAC, TAA, TAG, CAT, CAC, CAA, CAG, AAT, AAC, AAA, AAG, GAT, GAC, GAA, GAG, TGT, TGC, TGA, TGG, CGT, CGC, CGA, CGG, AGT, AGC, AGA, AGG, GGT, GGC, GGA, or GGG. In some embodiments, at least one or more of the determined end sequences comprises TGT, GAG, GCG, or ATT.

In some embodiments, the first frequency of the determined end sequence is indicative of the presence of a somatic mutation when the first relationship is indicative of the disease. In some embodiments, the first frequency of the determined end sequence is indicative of the presence of a copy number variation when the first relationship is indicative of the disease.

In some embodiments, the first cfDNA sample can be obtained from a biological sample from the first subject. In some embodiments, the second cfDNA sample can be obtained from a biological sample from the second subject.

In some embodiments, the disease is a cancer. In some embodiments, the cancer is a cancer of the central nervous system. In some embodiments, the cancer is a metastatic lesion. In some embodiments, the cancer is selected from bladder cancer, breast cancer, cervical cancer, colorectal cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, the cancer is a pancreatic cancer, lung cancer, or colorectal cancer.

### EXAMPLES

The disclosure is further described in the following examples, which do not limit the scope of the disclosure described in the claims.

### Library Preparation and Sequencing

Duplex libraries were prepared, wherein barcoded libraries were sequenced on Illumina HiSeq 4000 or Novaseq 6000 to an average depth of 18 million molecules. Adapters were then trimmed using cutadapt and trimmed sequences were aligned to the hg19 genome using samtools in paired end mode. Reads were then filtered for a MAPQ>1 and duplicates were removed using unique molecular identifiers (UMIs).

### Fragment End Analysis

For every unique molecule the first (5′) and last three (3′) nucleotides of the fragment were evaluated and the frequency of each motif at each end was determined. Understanding that library preparation either degrades or extends the 3′ end of the original DNA duplex to match that of the 5′ of the opposite strand averaged the 5′ motif frequency was averaged with the 3′ frequency of the reverse complement.

$$Frequency ( {motif}_{i} ) = \frac{( {N}_{{motif}_{i} , 5 prime} + {N}_{reverse {complement}_{i} , 3 prime} ) / 2}{{N}_{fragments}}$$

To determine end-motif frequencies within GC bins each fragment was binned into bins of 1% based on the GC content of the insert sequence. Next, the frequency of each end-motif was determined within each GC bin for a total of 3840 different frequencies.

$$Frequency ( {motif}_{i} , {bin}_{j} ) = \frac{( {N}_{{motif}_{i} , 5 prime , {bin}_{j}} + {N}_{reverse {complement}_{i} , 3 prime , {bin}_{j}} ) / 2}{{N}_{{bin}_{j}}}$$

### Classification Model

For classification of samples two approaches were taken. In the first approach random forest classifiers were trained using genome-wide frequencies of the 64 possible 3 bp end-motifs. In the second approach fragments were separated into bins based on their GC content and 3 bp end-motif frequencies were determined for each bin for a total of 3840 features. For both models cross-validation was performed by splitting the data into 80% training and 20% testing (5-fold) and scaled using StandardScaler. Scaled feature values were input into logistic regression models using sklearn and default parameters. This process was repeated ten times and the average scores were used to determine overall performance.

### Feature Selection

Feature selection was performed within each round of cross-validation. During each fold of cross-validation the samples within the training set were used to evaluate the mutual information between each feature and cancer status as well as the Mann-Whitney p-value for feature values of healthy controls vs. cancer samples. Thresholds were selected to be a specific percentile based on these values and only features that exceeded a given threshold were used for training. This process was repeated within every fold of cross-validation.

### Example 1-Fragmentation Patterns on Fragment Ends of cfDNA

This study evaluated 20 advanced colorectal cancer patients in whom circulating tumor DNA represented a major fraction of the total cfDNA and 37 healthy individuals of similar age (Cohort 1) Whole genome sequencing was performed on the cfDNA of each of these patients. Two key observations about the sequences of the bases at the ends of fragments were made during this initial evaluation, which led to evaluation of more patients and developing algorithms for classifying cancer patients based on the evaluations. Result on the trimers at both 5′-ends of DNA fragments are described herein, although similar observations were made about dimers, tetramers, etc. As used herein, “trimers” can refer to the 3 bases at the 5′-ends of fragments observed in cfDNA from plasma.

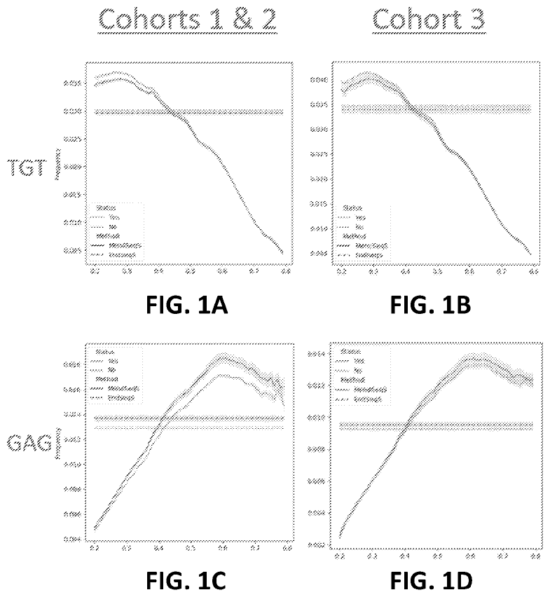

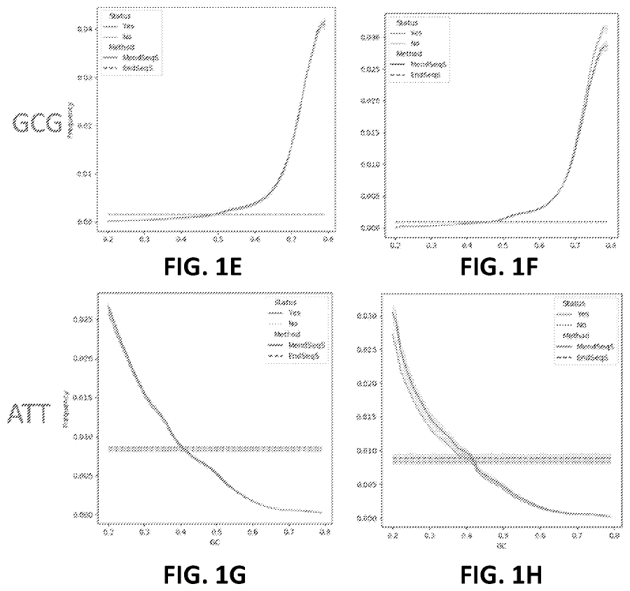

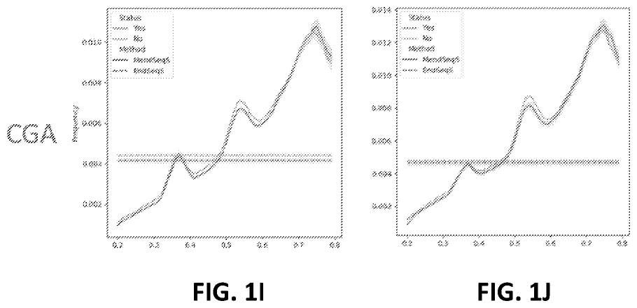

It was shown that the frequencies of trimers at the 5′-ends of fragments varies with the GC content of the entire fragment (i.e., the 3 base pairs at each of the two ends plus the 70 to 400 nucleotides between the ends) and follow four general trends depending on the four possible GC contents of the trimer. Trimers of middle GC content (2 A/T or 2 G/C) showed a linear correlation with fragment GC content while trimers of extreme GC content (3 A/T or 3 G/C) showed an exponential correlation with fragment GC content (FIGS. 1A-1J). This observation was made in samples derived from healthy individuals as well as from cancer patients and reproduced in publicly available data. FIGS. 1A-1B shows an example of one particular trimer (TGT) for data gathered for this study (FIG. 1A, Cohorts 1 and 2) and from public sources (FIG. 1B, Cohort 3). In this case, fragments ending in TGT are preferentially (y-axis) derived from fragments with lower GC content (x-axis) than from fragments with higher GC content. For example, at a GC interval of 74% to 75%, fragments ending in CGA account for only 0.5% of the total fragments in that GC interval but 3.5% at the GC interval between 25% and 26% (FIGS. 1I-1J). FIGS. 1C-1D show an example of another trimer (GAG) with an inverse pattern to that of TGT. Fragments ending in GAG are less often derived from fragments with low GC content than from fragments with higher GC content. FIGS. 1E-1F and FIGS. 1G-1H illustrate the trends of two trimers with extreme GC content, GCG (100% G/C) and ATT (100% A/T), respectively.

### Example 2-Cancer-Specific Differences is Dependent on GC Content

The magnitude of cancer-specific differences in trimers at the 5′-ends of fragments was shown to be dependent on the GC content of the entire cfDNA fragment. It has been demonstrated that the frequencies of trimers at the 5-ends of fragments derived from healthy individuals is different than those derived from patients with cancer. It was observed in this study that these cancer-specific differences are substantially more pronounced if GC content of the entire fragment is taken into account.

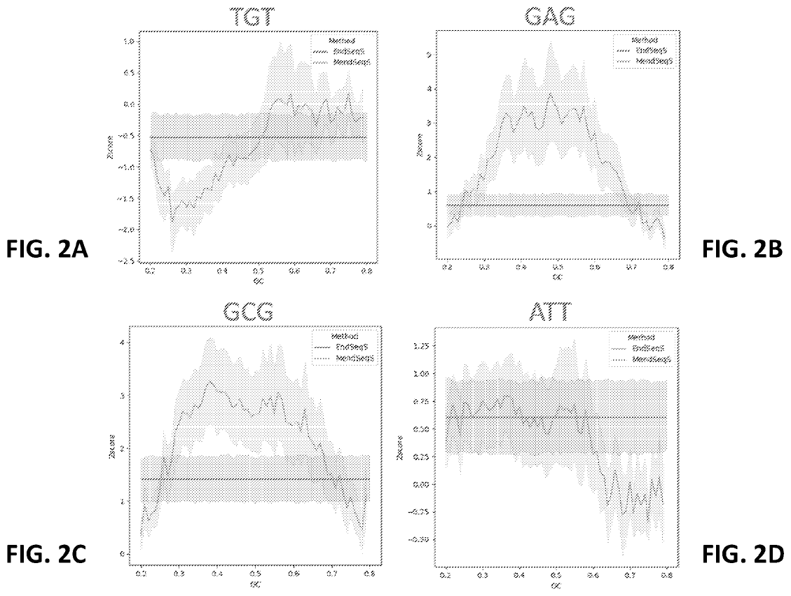

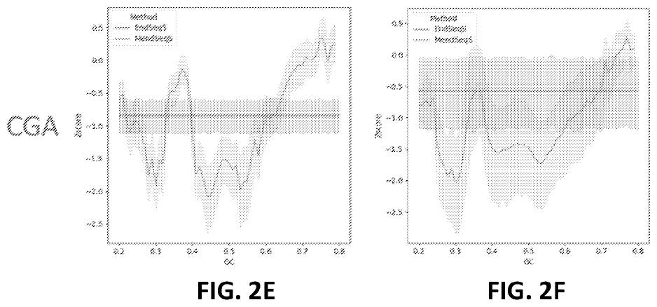

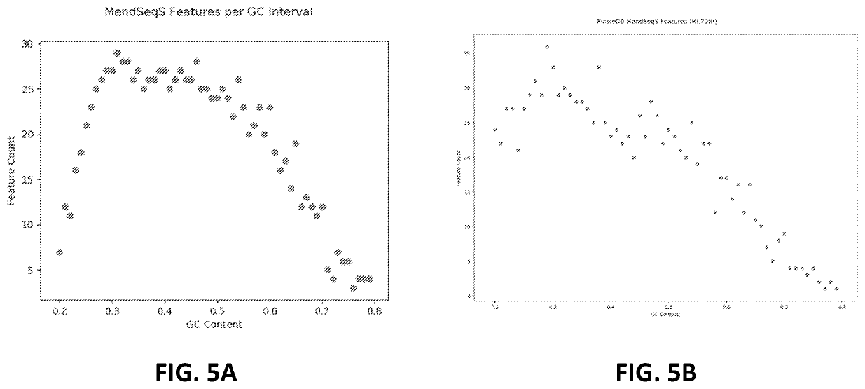

FIGS. 2A-2F illustrates this observation by measuring the difference in trimer frequency between cancer samples and healthy controls using the same trimers shown in FIGS. 1A-1F. For each trimer, a conventional z-score was calculated based on the frequencies of the trimer relative to that of the healthy controls in Cohort 1 for unadjusted (univariate) trimer frequency and GC-binned (conditional) trimer frequency. For TGT, the univariate z-score was −0.5 (blue horizontal line in FIG. 2A) while the conditional z-score varied depending on GC content but had a minimum z-score of −1.8 at 26-27% GC. Furthermore, the Mann-Whitney p-value between cancer and healthy was significant higher for this GC interval when compared to that for the univariate frequency (5.011e-11 and 0.00416, respectively) (FIGS. 5A-5B). The z-scores distinguishing cancer from normal samples in this example were clearly dependent on the GC content of the entire fragment. At relatively low GC content, there was significant difference between the TGT z-scores from patients with or without cancers and a marked improvement compared to the univariate z-score. At high GC content, however, there was relatively little difference in TGT frequency between patients with and without cancer.

Trimers GAG and GCG displayed a different pattern (FIGS. 2B-2C). At relatively low or high GC content were relatively low z-score and no improvement compared to the univariate trimer frequency. On the other hand, at moderate GC content between 35% and 55% there was a large difference between z-scores from patients with or without cancers, with z-scores>3-substantially higher than the univariate z-scores (1.4 and 0.76 for GAG and GCG, respectively) when GC content was not taken into account.

FIG. 2D illustrates a trimer (ATT) in which there was relatively little change in z-score conditional on GC content nor was there a significant change when comparing the univariate (i.e. without taking GC into account) and conditional z-scores. In this case, there were cancer-specific differences between the 5′ ends of fragments, regardless of GC content of the entire fragment. The average univariate z-score similar to that of most of the 60 GC-dependent Z-scores. FIGS. 2E-2F illustrates the patterns for the trimer CGA.

### Example 3-Trimers Exhibiting GC-Dependent, Cancer-Specific Differences

Feature selection based on the two observations described above in Examples 1 and 2, it was clear that a subset of trimers exhibited GC-dependent, cancer-specific differences in frequencies. Therefore, it was sought to construct a new classifier that exploited these observations. To incorporate the relationship between GC content and trimer frequency into the model used herein, the conditional probability of each trimer was first calculated given a fragment of a particular GC content, P(Trimer|Fragment GC), for each sample. In order to reduce the number of redundant features and increase performance feature selection was next performed on the set of 3840 possible features (64 trimers×60 GC intervals). For each feature data from Cohort 1 was used (average of 0.8× coverage) to compute (1) the mutual information between feature values and cancer status and (2) a Mann-Whitney p-value between feature values in cancer patients and healthy individuals.

Next, the data was filtered for features with mutual information and/or p-values above a specific threshold. To test the performance of each feature set data from Cohort 1 was used to train a logistic regression model on each set and measured performance of the model using cross-validation. It was found that the optimal number of features was 1152, representing 47 trimers and all 60 GC intervals that that were most informative for distinguishing cancer from normal samples in Cohort 1. This model was frozen and used for downstream testing. The Cohort used for the training set of this classifier was the same one used in to make the observations described above, i.e., 20 samples from cancer patients and 37 samples from healthy individuals (Cohort 1). This basic approach—feature selection based on cancer-specific trimers and GC intervals derived from sequencing data—was named Modified End-based sequencing (“MendSeq”).

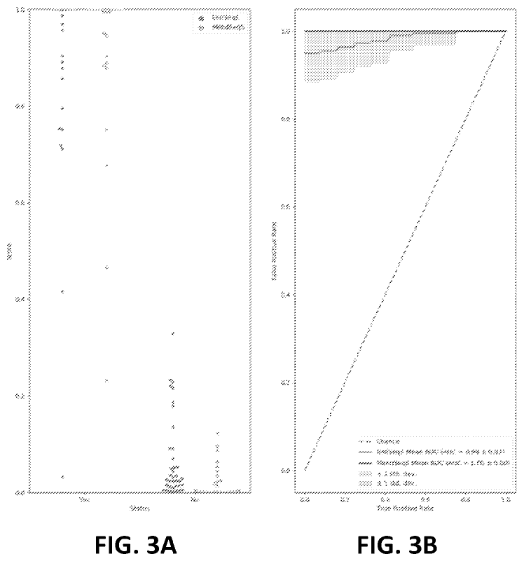

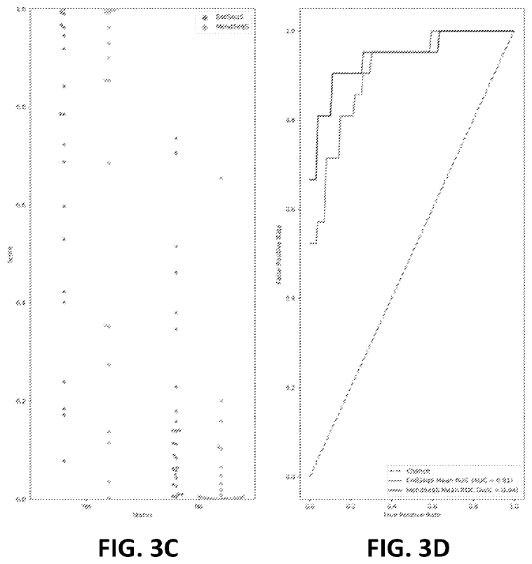

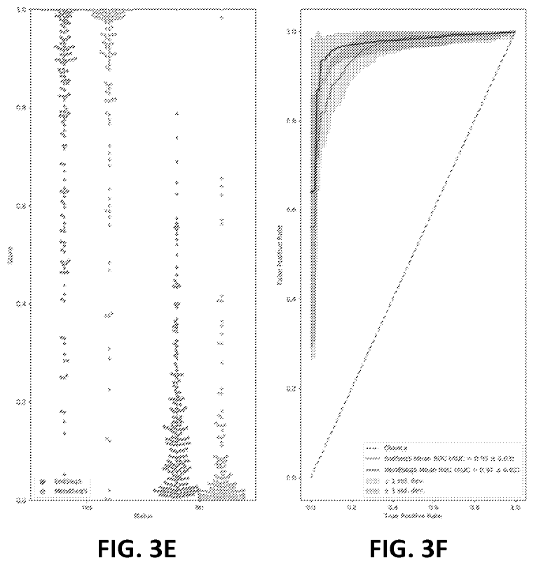

To employ these 1152 features in a classifier, logistic regression was used to assign weights (coefficients) to each of the 1152 features from the data on Cohort 1. This yielded a single MendSeq score for each patient in Cohort 1 (FIG. 3A). An ROC curve constructed from these data using cross-validation is shown in FIG. 3B, documenting a 100% sensitivity at 100% specificity and Area Under Curve (AUC) of 1.0±0.0. For comparison, each sample was scored in the identical way, but instead of using 1152 features, only 64 features were used—the univariate frequencies of each of the 64 trimers without considering GC content of the underlying fragments. This heuristic was named EndSeq and the resultant ROC curve is plotted in FIG. 3B.

To assess the performance of MendSeq in an independent set of samples, whole genome sequencing (WGS) (average of 1× coverage) was performed on another cohort of patients (Cohort 2). There were 27 healthy controls in Cohort 2 and 21 patients with cancer, the majority of which had advanced colorectal cancers, similar to those in Cohort 1. MendSeq was used to score each of the Cohort 2 patients using the 1152 features and logistic regression coefficients derived from Cohort 1. Dot plots of the scores and the derived ROC curves are plotted in FIGS. 3C-3D, respectively. The ROC curve for Cohort 2 (FIG. 3D) was similar to that of Cohort 1 (FIG. 3B), with a 67% sensitivity at 100% specificity, 76% sensitivity at 95% specificity, and an overall AUC of 0.942. This classification performance was significantly better than that achieved with EndSeq, for which the sensitivity was 52.3% at 100% specificity, 57.1% sensitivity at 95% specificity, and an overall AUC of 0.908 (FIG. 3D).

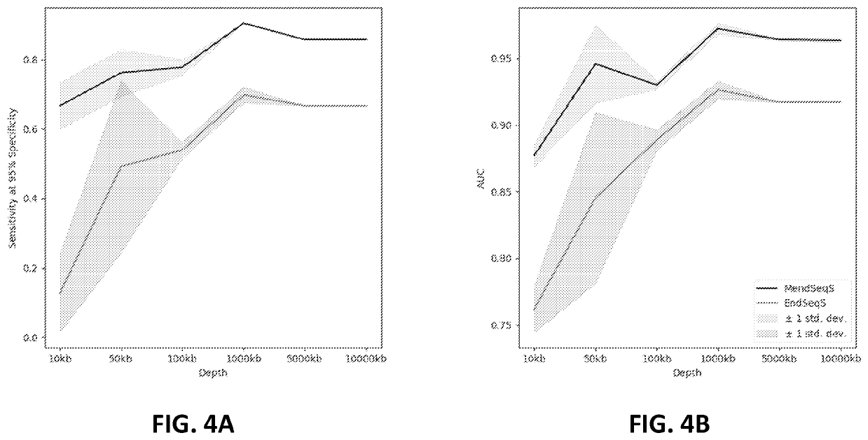

The DNA samples used to in Cohort 1 and Cohort 2 had an average depth of 17 million (˜0.8×) and 21 million reads (˜1×), respectively. To determine the effect of sequencing depth required on classification, the WGS data was down-sampled, randomly selecting 10 thousand to 10 million reads from the original WGS data. This process was repeated three times and the average sensitivity at 95% is illustrated in FIG. 4A while the average AUC is displayed in FIG. 4B. It was observed that MendSeq could achieve sensitivities >65% with a specificity of 95% achieved with as few as 10,000 reads. Comparatively, EndSeq had significantly lower performance at all depths.

It was then sought to see if MendSeq could be applied to DNA samples that had been prepared, amplified, and sequenced in other laboratories. For this purpose, 352 samples deposited in the FinaleDB database (called Cohort 3) were employed. These samples were donated by 208 healthy individuals and 144 cancer patients. The cancers represented in Cohort 3 were quite different than those in Cohorts 1 and 2, consisting of 23 colon, 43 pancreatic, and 78 lung tumors with most being early stage rather than advanced. The WGS data for Cohort 3 were generated by three different laboratories, each using different technologies.

The first question addressed was whether the two basic observations that formed the rationale for MendSeq were apparent in Cohort 3. FIGS. 1B, 1D, 1F, 1H, and 1J show the GC-dependence of the frequencies of same 3 trimers illustrated in FIGS. 1A, 1C, 1E, 1G, and 1I, but from Cohort 3 rather than Cohort 1. It was obvious from these data that there is a strong GC-dependence of trimer frequency and a clear improvement in the distinction between cancer vs. healthy individuals if GC content of the entire fragment is taken into consideration (FIGS. 2A-2F) in Cohort 3.

The second question was whether implementation of MendSeq could improve the classification of cancer samples over that achieved with Unadjusted WGS in Cohort 3. As in Cohort 1, mutual information and Mann Whitney p-values were used to select 1152 of the potential 3840 features available for MendSeq-based classifier. These features included every GC interval (60) and every trimer (64). With Cohort 3, however, stage information for the cancer patients was unavailable (unlike Cohort 1), so unsupervised cross-validation instead of supervised selection was used to choose the features and logistic regression coefficients for each features were determined for each round of cross-validation. Ten rounds of 5-fold cross validation were used.

Dot plots of the scores in Cohort 3 derived from MendSeq are plotted in FIG. 3E and ROC curves for both MendSeq and EndSeq are plotted in FIG. 3F. With MendSeq, the sensitivity was 63.9% at 100% specificity, 93.6% at 95% specificity, and an overall AUC of 0.971±0.02. This classification performance was significantly better than that achieved with EndSeq, for which the sensitivity was 56.2% at 100% specificity, 81.9% at 95% specificity, and an overall AUC of 0.953±0.03 (FIG. 3F).

### Example 4-MendSeqS Optimized Method Workflow

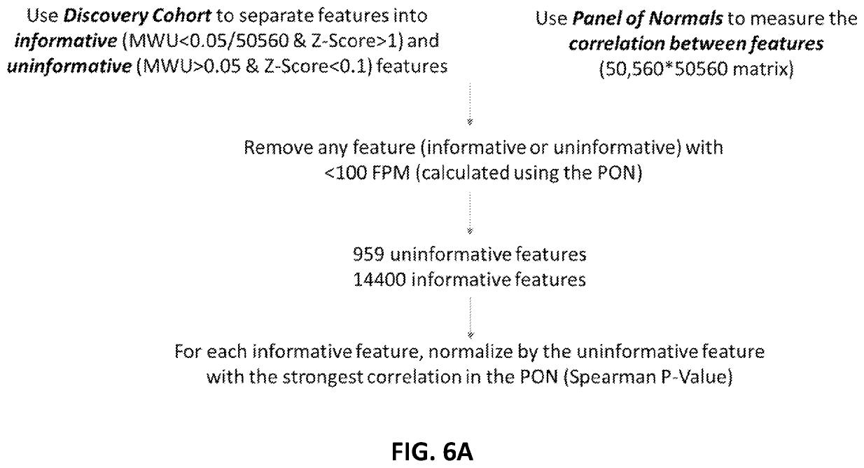

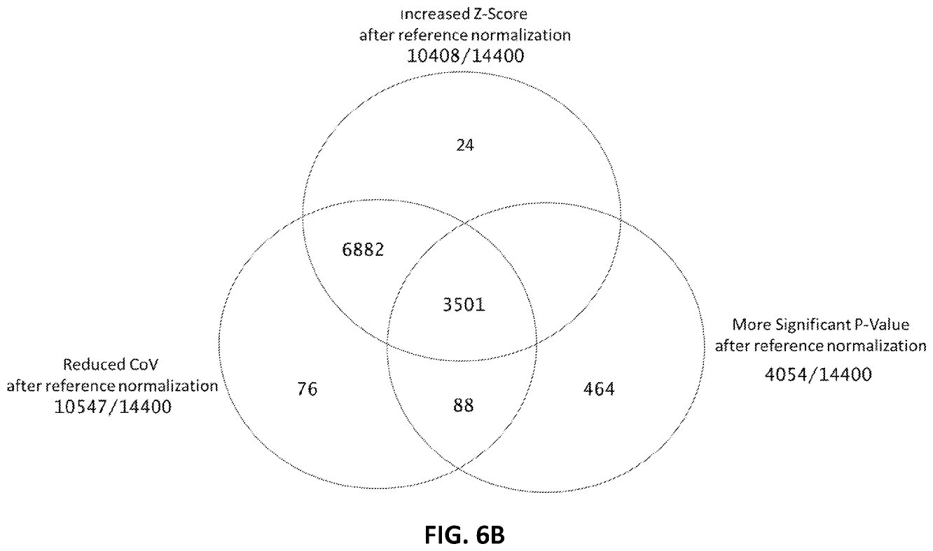

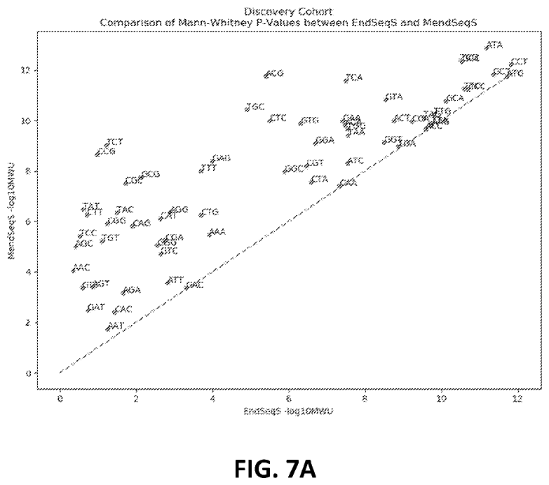

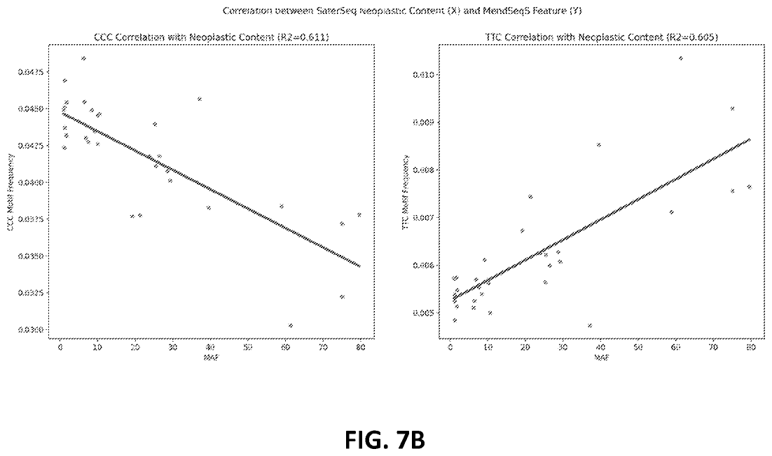

A normalization method (e.g., “Reference Normalization”) was developed to decrease technical noise in MendSeqS data (FIGS. 6A-6B), wherein it was discovered that MendSeqS features are strongly correlated with neoplastic content in the plasma. SaferSeqS was used to analyze MendSeqS features in molecules that are derived from the tumor (circulating tumor DNA), and information from SaferSeqS mutant molecules was incorporated into feature selection to select for features that are tumor-derived (FIGS. 7A-7B). Furthermore, rigorous feature selection was incorporated to remove batch effects, decrease overfitting, reduce dimensionality of features, and increase confidence in the model. The results illustrated that MendSeqS can effectively detect early and late-stage cancers using a small set of only three features (out of a possible 50,560).

For every individual sample, the occurrence of fragment-end motifs in circulating-free DNA molecules of a given patient sample was counted. The counts of each end-motif were distributed into specific ‘GC bins’ depending on the GC content of the molecule the motif was derived from. These bins can be varied in size (e.g. 20-21% GC, 20-25% GC, 20-50% GC). After analyzing every molecule in a given patient sample, the frequency of each motif in each GC bin was calculated (e.g. what is the frequency of the motif AAA in molecules with 20-25% GC content?).

After analyzing many samples, and using only samples from individuals without cancer (e.g., “Panel of Normals”), the noise of each feature was calculated by looking at the standard deviation of the feature value (frequency) between many healthy individuals. How correlated each feature is with every other feature was calculated to generate a correlation heatmap, wherein a variety of statistical tests can be used for this step (e.g., a PearsonR or SpearmanR).

Using samples from patients with cancer and individuals without cancer, the ability of each feature to distinguish patients with cancer from healthy individuals was calculated by comparing the feature value (frequency) of the feature in individuals with cancer to those without cancer, wherein a variety of statistical tests can be used for this step (e.g. Z-Scores, Mann-Whitney U Test, Students T-Test).

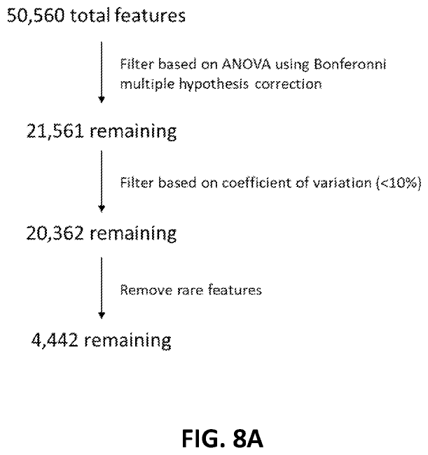

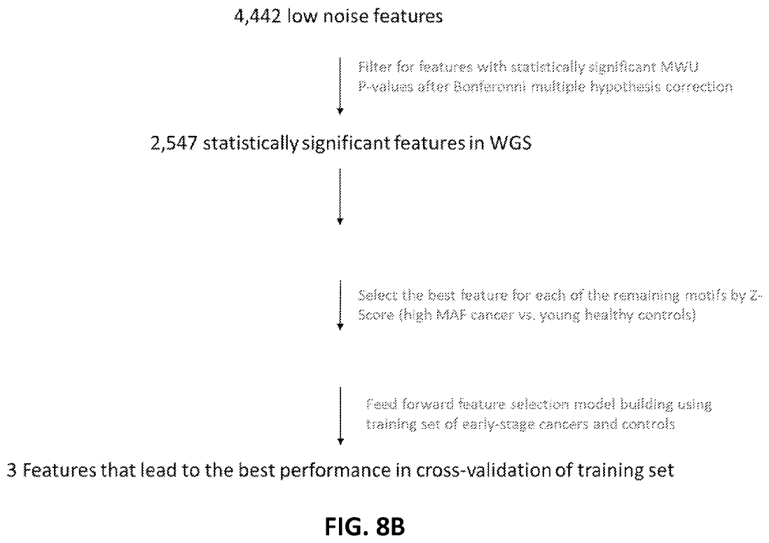

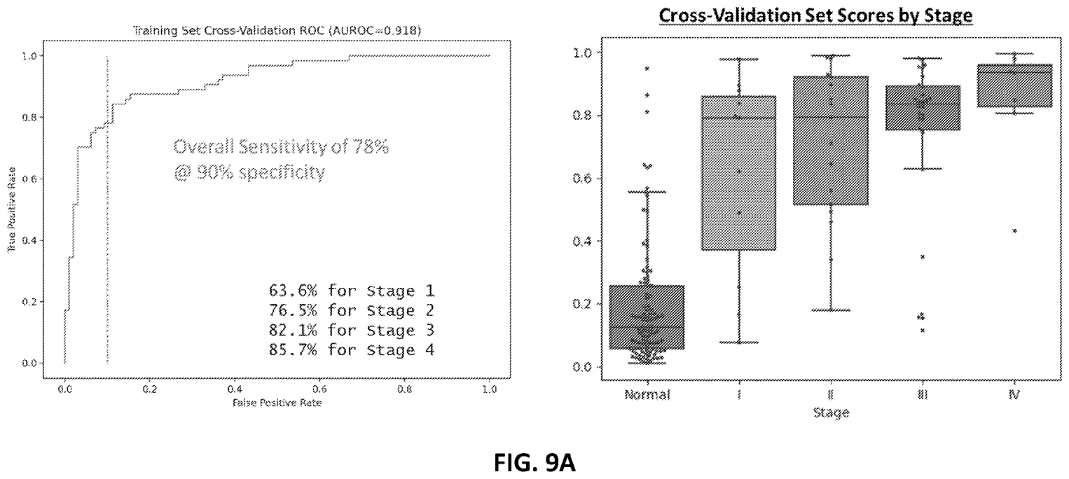

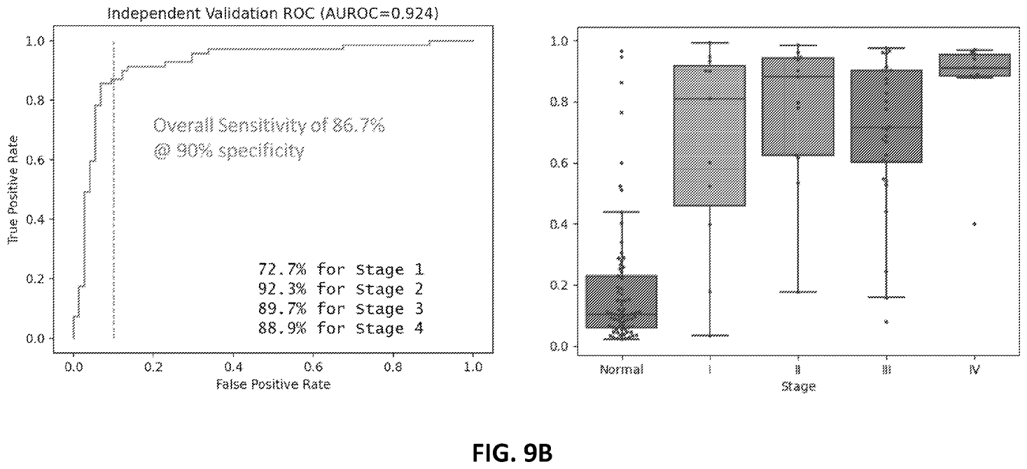

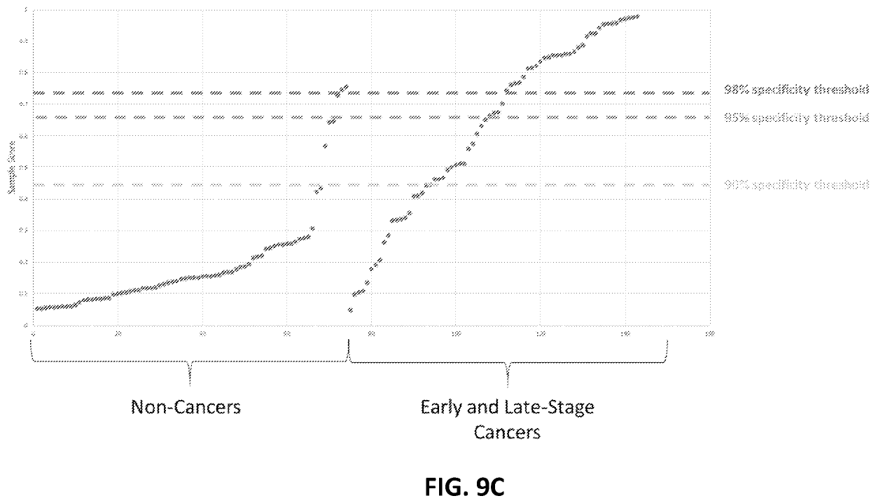

Using information gathered as described above, a set of features were selected that are known to be informative for distinguishing patients with cancer from healthy individuals. This can be done with multiple hypothesis correction (e.g. Bonferonni, Benjamini-Hochberg). A set of features that are known to be uninformative were also selected for distinguishing patients with cancer from healthy individuals. For each informative feature, the uninformative feature that it is most correlated with was selected. The frequency of the informative feature was then divided by the uninformative feature to get a normalized value. These steps were repeated using normalized data, wherein a small subset of highest signal: noise features were selected based on the calculated statistics (FIGS. 8A-8B). Using these features, a machine learning (ML) model was trained to distinguish between patients with cancer and healthy individuals (FIGS. 9A-9C).

What is claimed is:

1. A method of identifying a subject as having a disease, the method comprising:
(a) obtaining a biological sample from the subject, wherein the biological sample comprises cell-free DNA (cfDNA), wherein the cfDNA comprises a plurality of cfDNA fragments;
(b) determining an end sequence of a cfDNA fragment of the plurality of cfDNA fragments;
(c) determining a level of GC content of the cfDNA fragment; and
(d) analyzing the determined end sequence and the level of GC content of the cfDNA fragment, thereby identifying the subject as having the disease by determining a relationship between the determined end sequence and the level of GC content of the cfDNA fragment.

2. The method of claim 1, wherein the cell-free DNA comprises double stranded DNA.

3. The method of claim 1 or 2, wherein the determined end sequence is at the 5′-end of one or both strands of the cfDNA.

4. The method of any one of claims 1-3, wherein the determined end sequence is 2, 3, 4, 5, or 6 bases in length.

5. The method of any one of claims 1-4, wherein the determined end sequence is 3 bases in length.

6. The method of any one of claims 1-5, wherein the determined end sequences comprises TGT, GAG, GCG, or ATT.

7. The method of any one of claims 1-6, wherein the relationship of the determined end sequence and the level of GC content of the cfDNA fragment from a subject having the disease is different as compared to the relationship of the determined end sequence and the level of GC content of the cfDNA fragment from a subject that does not have the disease.

8. The method of any one of claims 1-7, wherein the biological sample is a plasma sample.

9. The method of any one of claims 1-7, wherein the biological sample is a cerebrospinal fluid (CSF) sample.

10. The method of any one of claims 1-9, wherein the disease is a cancer.

11. The method of claim 10, wherein the cancer is a cancer of the central nervous system.

12. The method of claim 10, wherein the cancer is a metastatic lesion.

13. The method of claim 10, wherein the cancer is selected from bladder cancer, breast cancer, cervical cancer, colorectal cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

14. The method of claim 10, wherein the cancer is a pancreatic cancer, lung cancer, or colorectal cancer.

15. A method of identifying a relationship between an end sequence and a disease state in a subject, the method comprising:
(a) obtaining a first cfDNA sample from a first subject having a disease and a second cfDNA sample from a second subject that does not have the disease, wherein the cfDNA samples comprise a plurality of cfDNA fragments;
(b) determining an end sequence of a first cfDNA fragment of the plurality of first cfDNA fragments and determining a first level of GC content of the first cfDNA fragment;
(c) determining the same end sequence of a second cfDNA fragment of the plurality of second cfDNA fragments and determining a second level of GC content of the second cfDNA fragment;
(d) measuring a first frequency of the determined end sequence in the first cfDNA fragment and a second frequency of the same determined end sequence in the second cfDNA fragment;
(e) identifying a first relationship between the first frequency of the determined end sequence and the determined first level of GC content from the first subject;
(f) identifying a second relationship between the second frequency of the determined end sequence and the determined second level of GC content from the second subject; and
(g) determining that the first relationship is indicative of the disease and that the second relationship is not indicative for the disease state.

16. The method of claim 15, wherein a cfDNA fragment comprises double stranded DNA.

17. The method of claim 15 or 16, wherein the determined end sequence is at the 5′-end of one or both strands of the cfDNA fragment.

18. The method of any one of claims 15-17, wherein the determined end sequence is 2, 3, 4, 5, or 6 bases in length.

19. The method of any one of claims 15-18, wherein the determined end sequence is 3 bases in length.

20. The method of any one of claims 15-19, wherein the determined end sequence comprises TGT, GAG, GCG, or ATT.

21. The method of any one of claims 15-20, wherein the first frequency of the determined end sequence is indicative of the presence of a somatic mutation when the first relationship is indicative of the disease.

22. The method of any one of claims 15-21, wherein the first frequency of the determined end sequence is indicative of the presence of a copy number variation when the first relationship is indicative of the disease.

23. The method of any one of claims 15-22, wherein the disease is a cancer.

24. The method of claim 23, wherein the cancer is a cancer of the central nervous system.

25. The method of claim 23, wherein the cancer is a metastatic lesion.

26. The method of claim 23, wherein the cancer is selected from bladder cancer, breast cancer, cervical cancer, colorectal cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

27. The method of claim 23, wherein the cancer is a pancreatic cancer, lung cancer, or colorectal cancer.
